(19) United States
(12) Patent Application Publication
(10) Pub. No.: US 2014/0309968 A1
(43) Pub. Date: Oct. 16, 2014

(54) SYSTEMS AND METHODS FOR PROCESSING FOOD ASSESSMENT DATA

(71) Applicant: Allripe Pty Ltd, Coogee (AU)

(72) Inventors: ERICA FAUSTINE DIEZMOS, South Hurstville (AU); Kallol Shah, Mumbai (IN); Kevin Lerdwichagul, Pagewood (AU); Dirk Anthony/Samuel Hertfford, Coogee (AU)

(73) Assignee: Allripe Pty Ltd, Coogee (AU)

(21) Appl. No.: 14/250,917

(22) Filed: Apr. 11, 2014

(30) Foreign Application Priority Data
Apr. 12, 2013 (AU) 2013901270

(51) Int. Cl. G01N 33/02

(52) U.S. Cl. CPC G01N 33/02
USPC 702/189

(57) ABSTRACT

In accordance with an aspect of the present invention, there is provided a computer implemented method (100) for processing food assessment data. The method includes the initial step (102) of providing an interface for enabling input of food assessment data. At step (104) a database is maintained for storing the input assessment data. At step (106) a processor is configured to analyse at least a subset of the assessment data based on one or more of the type and source of the assessment data to produce a food assessment model indicating the safety and/or quality of a selected one or more food items.

### FIELD OF THE INVENTION

The present invention relates to computer implemented systems and methods for processing food assessment data. Embodiments of the invention have been particularly developed for enabling users to input food assessment data and obtain a quality assessment of food items. While some embodiments will be described herein with particular reference to that application, it will be appreciated that the invention is not limited to such a field of use, and is applicable in broader contexts.

### BACKGROUND

Any discussion of the background art throughout the specification should in no way be considered as an admission that such art is widely known or forms part of common general knowledge in the field.

At present, it is difficult for the general public to affordably, accurately and easily assess pieces of food for quality and safety. Traditional programs available for food assessment are linked to a narrow range of expensive specialist equipment and are difficult for non-specialists to utilise. These programs do not encompass a broad range of data sources, nor do they include user feedback based on human senses. Consequently, only limited information can be obtained on the quality and safety of food items, and generally only at a relatively high cost.

There is a need in the art for improved systems and methods for analysing food assessment data.

### SUMMARY OF THE INVENTION

It is an object of the present invention to overcome or ameliorate at least one of the disadvantages of the prior art, or to provide a useful alternative.

According to a first embodiment of the present invention there is provided a computer implemented method to process food assessment data, the method including:
a) providing an interface to enable input of food assessment data;
b) maintaining a database to store the assessment data;
c) configuring a processor to analyse the state of at least a subset of the assessment data based on one or more of the type and source of the assessment data to produce a food assessment model indicating the safety and/or quality of a selected one or more food items.

The processor is preferably also configured to identify the type of input food assessment data and, in response, convert the data to a second data format that is independent of the data type. The processor is preferably also configured to identify the source of input food assessment data and, in response, convert the data to a second data format that is independent of the data source.

The processor is preferably configured to assign attributes to the input food assessment data based on the type and source of the data.

The processor preferably separately analyses the assessment data based on a type and/or source of the data. The type of the data preferably includes human sense data and instrument sensor data. The source of the data preferably includes human input or electronic sensor input.

In one embodiment, the method includes the step of:

c) i) calculating one or more food assessment parameters.

The one or more food assessment parameters preferably includes the Edibility(E), Freshness(F), Longevity(L), Ripeness(R) or Safeness to eat(S) of the food item.

In one embodiment, the method includes the step of:

c) ii) comparing the one or more food assessment parameters with a corresponding benchmark for that particular source or type.

In one embodiment, the method includes the step of:
d) iii) combining the comparisons of the one or more food assessment parameters for each data source and type.

The processor preferably applies a numerical weighting to the food assessment data based on the data attributes.

The food assessment model preferably includes one or more ratings indicative of the relative quality or safety of the one or more food items. The one or more ratings are preferably numerical ratings.

The input food assessment data preferably includes raw data received from measuring instruments. The input data source preferably includes electronic sensors and detectors.

The input food assessment data preferably includes human observation data.

The input data preferably includes information indicative of one or more of a food's visual appearance, smell, size/volume, taste, flavour, colour, ripeness and firmness.

The input data type preferably includes text input, photographs, video, sensor data and spectrometer data. Data can also be collected from refrigerators and other appliance sensors. This appliance data can include temperature, humidity and time spent in appliance.

The food assessment model preferably includes an assessment of whether a food is safe to eat. The food assessment model preferably includes an assessment of the freshness of a food. The food assessment model preferably includes an assessment of the longevity of a food.

The food assessment model is preferably associated with an inline frame for allowing the model to be embedded within a website. The food assessment model preferably includes an assessment report adapted to be shared through one or more social media websites.

In accordance with a second aspect of the present invention there is provided a computer system configured to perform a method according to the first aspect of the invention.

In accordance with a third aspect of the present invention there is provided computer program configured to perform a method according to the first aspect of the invention.

In accordance with a fourth aspect of the present invention there is provided a non-transitive carrier medium carrying computer executable code that, when executed on a processor, causes the processor to perform a method according to the first aspect of the present invention.

In accordance with a fifth aspect of the present invention there is provided a computer system to process food assessment data, the system including:
an interface to enable input of food assessment data;
a database to store the assessment data;
a processor configured to analyse the state of at least a subset of the assessment data based on one or more of the type and source of the assessment data to produce a food assessment model indicating the safety and/or quality of a selected one or more food items.

In accordance with a sixth aspect of the present invention there is provided a computer implemented method to provide food assessment data to a user, the method including:
a) providing an interface to enable users to input individual assessments of a food item;
b) maintaining a database to store the individual assessments;
c) configuring a processor to produce an assessment model of the food item based on a combination of the individual assessments.

In accordance with a seventh aspect of the present invention there is provided a computer implemented method to provide food assessment data, the method including:
a) providing an interface to enable one or more users to input user assessments of one or more food items;
b) providing a communications module to receive device assessment data from measurement devices;
c) maintaining a database to store the user assessments and device assessment data;
d) upon a request by a user, configuring a processor to produce an assessment model of a preselected food item, the model being based on a combination of the user assessments and device assessment data for the preselected food item.

In accordance with an eighth aspect of the present invention there is provided a computer implemented method to provide an indication of one or more characteristics of a sample food item, the method including:
a) providing an interface to enable the creation of a sample record for the sample food item, the record including either or both of quantitative or qualitative data for the sample food item;
b) maintaining a database to store the sample record and other records for respective other sample food items; and
c) configuring a processor to implement a food assessment model that is selectively responsive to the sample record and the other records to provide an indication of one or more characteristics of the sample food item.

In one embodiment, the sample food item is a virtual food item.

In accordance with a ninth aspect of the present invention there is provided a system to provide an indication of one or more characteristics of a sample food item, the system including:
an interface to enable the creation of a sample record for the sample food item, the record including either or both of quantitative or qualitative data for the sample food item;
a database to store the sample record and other records for respective other sample food items; and
a processor to implement a food assessment model that is selectively responsive to the sample record and the other records to provide an indication of one or more characteristics of the sample food item.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

### BRIEF DESCRIPTION OF THE DRAWINGS

Embodiments of the invention will now be described, by way of example only, with reference to the accompanying drawings in which:

FIG. 1 illustrates an exemplary process flow of a method of analysing food assessment data according to an embodiment of the invention;

FIG. 2 illustrates an exemplary client server arrangement for implementing a method according to an embodiment of the present invention;

FIG. 3 illustrates an exemplary process flow of a user uploading food assessment data;

FIG. 4 illustrates a screenshot of a first exemplary webpage for uploading food assessment data from a personal computer;

FIG. 5 illustrates a screenshot of a second exemplary webpage for uploading food assessment data from a personal computer;

FIG. 6 illustrates a screenshot of a third exemplary webpage for uploading food assessment data from a personal computer;

FIG. 7 illustrates a screenshot of a fourth exemplary webpage for uploading food assessment data from a personal computer;

FIG. 8 illustrates a screenshot of a fifth exemplary webpage for uploading food assessment data from a personal computer;

FIG. 9 illustrates four screenshots of an exemplary mobile application ‘App’ for uploading food assessment data from a mobile device;

FIG. 10 illustrates an exemplary process flow of a sorting algorithm;

FIG. 11 illustrates an exemplary look-up table for relating input raw frequency data to fruit ripeness and quality rating;

FIGS. 12A to 12C collectively illustrate the process flow of an algorithm performed by a processor to generate a composite food assessment model;

FIG. 13 illustrates schematically a processor with functionality as both an internal analysis engine and a combiner engine;

FIG. 14 illustrates an exemplary process flow of a user requesting an assessment model of food items;

FIG. 15 illustrates a graphical representation of a composite food assessment model in the form of a pie diagram

FIG. 16 illustrates an exemplary web page displaying an online food assessment report including a pie model graphic;

FIG. 17 illustrates a graphical representation of a composite food assessment model in the form of a map diagram;

FIG. 18 illustrates an exemplary web page displaying an online food assessment report including a map model graphic;

FIG. 19 illustrates a graphical representation of a composite food assessment model in the form of a line diagram;

FIG. 20 illustrates an exemplary web page displaying an online food assessment report including a line model graphic;

FIG. 21 illustrates a screenshot of an exemplary webpage displaying time-lapse results of assessment data.

### DETAILED DESCRIPTION

Described herein are computer implemented systems and methods for processing food assessment data. As used herein, the term “food assessment data” refers to data indicative of one or more characteristics of a sample food item. The food assessment data includes two main sources: qualitative user data, such as text input and photos uploaded by users; and quantitative field data, such as data measured in the field by sensors and instruments.

### General Overview

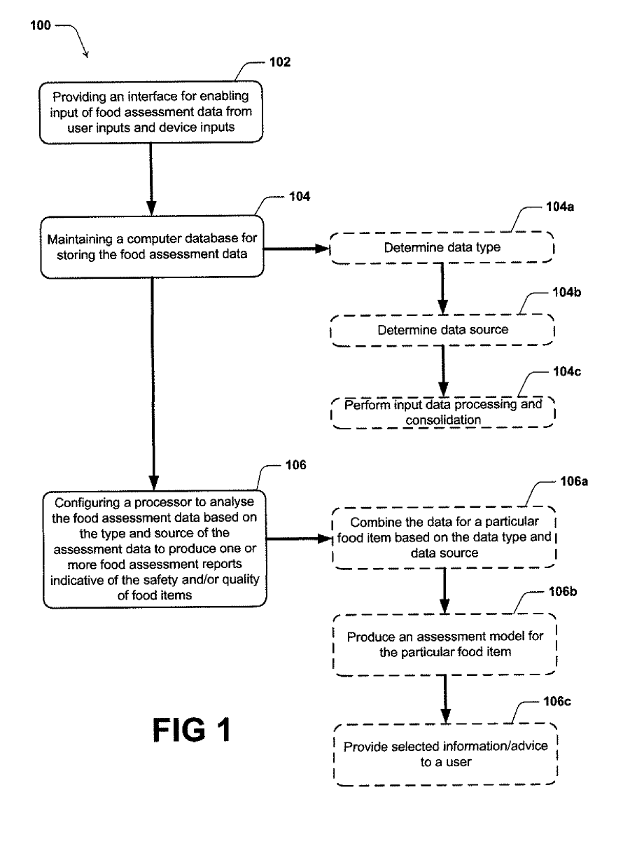

Embodiments of the invention described herein are particularly developed to enable the uploading of various forms of food assessment data, and the subsequent analysis of that data. Preferred embodiments described herein are particularly adapted for the assessment and analysis of fruit and vegetable type food items. However, it will be appreciated that, in other embodiments, various other food items are able to be assessed. Referring to FIG. 1, one embodiment of the invention provides a computer implemented method 100 for processing food assessment data. The method is particularly developed to be performed by a computer having at least an associated processor, database, input/output data communications interface (such as a network port or Wi-Fi controller), display interface and an interface for connecting user input devices such as a keyboard, mouse or touch screen.

At step 102 of method 100, an interface is provided for enabling input of food assessment data from user inputs such as food reviews and observations, and from instrument inputs such as test device readings. The interface enables the creation of a sample record for a sample food item. The record includes either or both of quantitative or qualitative data for the sample food item. At step 104 the database is maintained for storing the food assessment data, sample record and other records for respective other sample food items. Upon input and storage of the food assessment data, some data management sub-steps are performed. At sub-step 104a, the data type of the food assessment data is determined. Example data types include text input from users of the interface, uploaded photographs and video, and instrument and sensor data such as spectrometer readings. At sub-step 104b, the data source of the food assessment data is determined. Example data sources include sensors, detectors and measurement instruments, cameras and human senses (smell, taste, feel and appearance of the food) through user input devices. Data can also be collected from refrigerators and other appliance sensors. This appliance data can include temperature, humidity and time spent in appliance. At sub-step 104c data processing is performed on the input food assessment data of varying types and sources to consolidate the data into a common data format for analysis. In some embodiments, other data processing, such as data filtering, is performed at sub-step 104c.

At step 106, the processor is configured to analyse the stored food assessment data based on one or more of the type and source of the data to provide an indication of one or more characteristics of the sample food item. In some embodiments, the indication is in the form of one or more food assessment reports indicative of the safety and/or quality of a selected one or more food items. In analysing the food assessment data, at sub-step 106a, the data relating to a predetermined food item (or group of food items) is combined based on the data type and data source and, at sub-step 106b, an assessment model is produced for the food item. Details of how this model is produced are set out below under the heading ‘analysis of food assessment data’. The assessment model includes one or more characteristics relating to the food item for which a user can select to view in the assessment report, including quality information, safety information, previous user reviews and previous assessments of that food item. The food assessment model is selectively responsive to the sample record and the other records stored in the database to provide an indication of one or more characteristics of the sample food item. In the assessment model, certain data types or data sources may be given more or less emphasis or weighting depending on the food item being assessed. By way of example, in the case of an item of fruit being assessed, more weighting may be given to visual appearance, acidity levels and firmness. Based on a user request, at sub-step 106c, specific information and advice relating to the food item is provided to the user in a tailored assessment report.

### Exemplary System-Level Overview

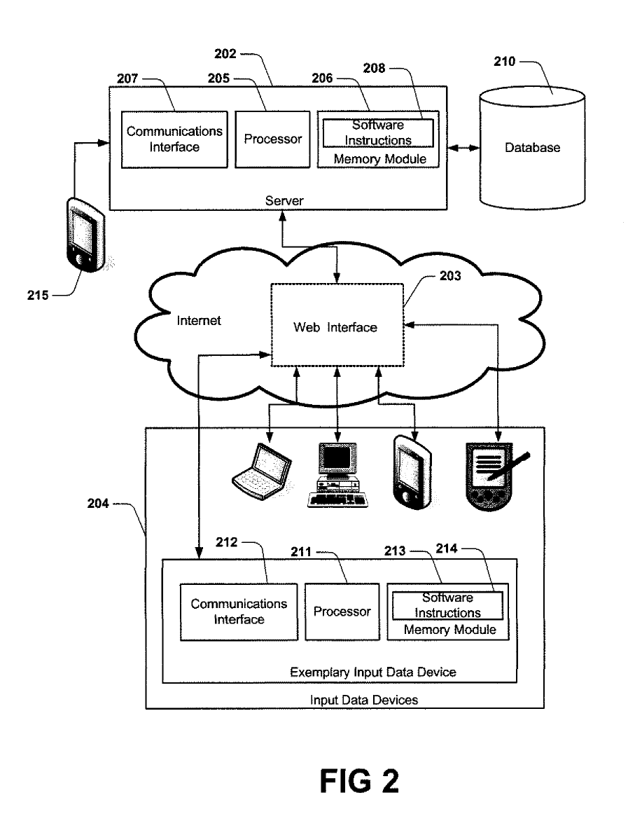

In some embodiments, methods and functionalities considered herein are implemented by way of a server, as illustrated in FIG. 2. In overview, a web server 202 provides a web interface 203. This web interface is accessed by the parties (users and system administrators) by way of client terminals 204. In overview, users access interface 203 over the Internet by way of client terminals 204, which in various embodiments include the likes of personal computers, PDAs, cellular telephones, gaming consoles, and other Internet enabled devices.

Server 203 includes a processor 205 coupled to a memory module 206 and a communications interface 207, such as an Internet connection, modem, Ethernet port, wireless network card, serial port, or the like. In other embodiments distributed resources are used. For example, in one embodiment server 202 includes a plurality of distributed servers having respective storage, processing and communications resources. Memory module 206 includes software instructions 208, which are executable on processor 205.

Server 202 is coupled to a database 210. In further embodiments the database leverages memory module 206.

In some embodiments web interface 203 includes a website. The term “website” should be read broadly to cover substantially any source of information accessible over the Internet or another communications network (such as WAN, LAN or WLAN) via a browser application running on a client terminal. In some embodiments, a website is a source of information made available by a server and accessible over the Internet by a web-browser application running on a client terminal. The web-browser application downloads code, such as HTML code, from the server. This code is executable through the web-browser on the client terminal for providing a graphical and often interactive representation of the website on the client terminal. By way of the web-browser application, a user of the client terminal is able to navigate between and throughout various web pages provided by the website, and access various functionalities that are provided.

Although some embodiments make use of a website/browser-based implementation, in other embodiments proprietary software methods are implemented as an alternative. For example, in such embodiments client terminals 204 maintain software instructions for a computer program product that essentially provides access to a portal via which assessment reports, models and assessment data are accessed (for instance via an iPhone app or the like).

In general terms, each terminal 204 includes a processor 211 coupled to a memory module 213 and a communications interface 212, such as an Internet connection, modem, Ethernet port, serial port, or the like. Memory module 213 includes software instructions 214, which are executable on processor 211. These software instructions allow terminal 204 to execute a software application, such as a proprietary application or web browser application and thereby render on-screen a user interface and allow communication with server 202. This user interface allows for the creation, viewing and administration of profiles, access to the internal communications interface, and various other functionalities.

### Exemplary Implementation

The creation of an assessment model is accomplished using software which is responsive to user inputs, and accesses database 210 and other online resources.

The software to generate a composite model of food assessments follows a number of stages. Each of these stages carry out different types of operation using particular inputs, either from the user or from database 210 or online resources which store information regarding different stages of a food (i.e. unripe, ripe, overripe). In addition, these databases also store ratings for different outcome parameters.

### Uploading of Food Assessment Data

Food assessment data is uploaded to server 202 and stored in database 210 by two primary processes: a) direct upload by assessment devices 215 used in the field (quantitative data); and b) upload by users who provide content through interface 203 accessed by client terminals 204 (qualitative data). The input data can take various forms, including instrument readings such as frequencies, numbers and codes in the form of electronic and/or digital signals, digital pictures, digital video footage, text descriptions, user sense-data, sense-impressions, template uploads. The various input data may or may not include abbreviations and corresponding units of measures.

In one embodiment, input data is characterised into outcome parameters, which are a set of variables including different attributes of a food and may contain a value depending on the condition of the food, say on a scale of 0 to 10. By way of example, the freshness of an apple can be user-rated by a value ranging from 0 to 10 with 10 representing maximum freshness.

Other outcome parameters which contain a single scale value include Edibility(E), Freshness(F), Longevity(L), Ripeness(R) and Safeness to eat(S). In addition to the above mentioned parameters, there is another parameter Model (M) which contains multiple variables containing both textual and numeric data. The variables include texture, colour, firmness, size, shape, weight as well as all the input variables from the user. The texture and shape variable may contain image patterns of a particular texture/shape which varies according to the food. The colour variable may contain the most prominent colours on the food based on red-green-blue (RGB) values. The size and weight variables will accept standard measurement and weight units. The firmness of the food can be on a scale of 0 to 10.

For upload process a), server 202 and/or database 210 are directly or indirectly accessible by assessment devices 215 for allowing quantitative food assessment data collected in the field to be transmitted to server 202 and database 210. Devices 215 include various instruments for assessing food quality and safety, and each include an integrated wireless device for wirelessly communicating with communications interface 207 to store the collected data in database 210. In other embodiments, devices 215 include communications ports for connecting to a personal computer or other communications device to transmit the collected data to server 202.

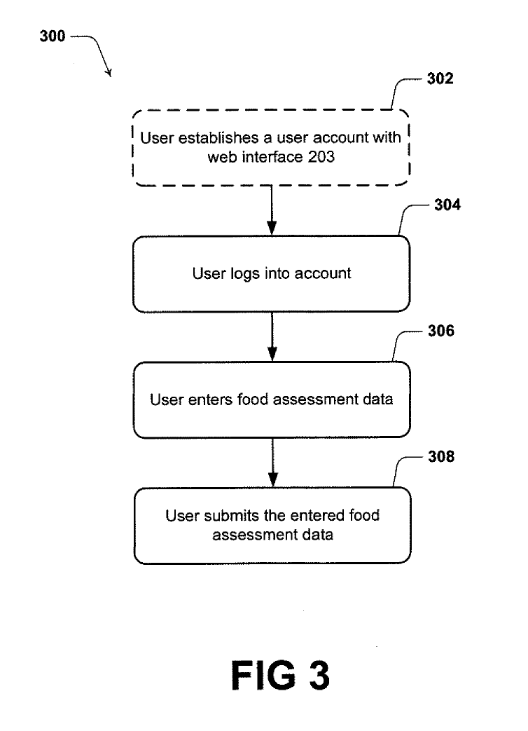

In upload process b), users follow the process 300 outlined in FIG. 3, as described below.

In accessing web interface 203, a user navigates to a particular host website having a particular domain name (for example, Allripe.com) through a web browser on one of client terminals 204. In another embodiment, web interface 203 is provided as a software App available for download to client terminals 204 such as Smartphones or tablets. Interface 203 presents users with fields and menus for inputting and uploading user data and qualitative food assessment data.

Referring to FIG. 3, at step 302, a user initially sets up a user account on interface 203 for repeat access to server 202, including providing a username, password and user type (e.g. non-specialist or specialist). The step of establishing a user account is only required to be performed once for a given user and the entered user data for an account is stored in database 210. The user account is used by administrators of server 202 for, amongst other things, monitoring user activity, statistical purposes, allowing communication between users, ranking users based on experience and activity and, in some embodiments, providing a reward scheme to users based on the amount of user activity. In other embodiments, a user account is not required and users are able to upload food assessment information on an ad-hoc basis.

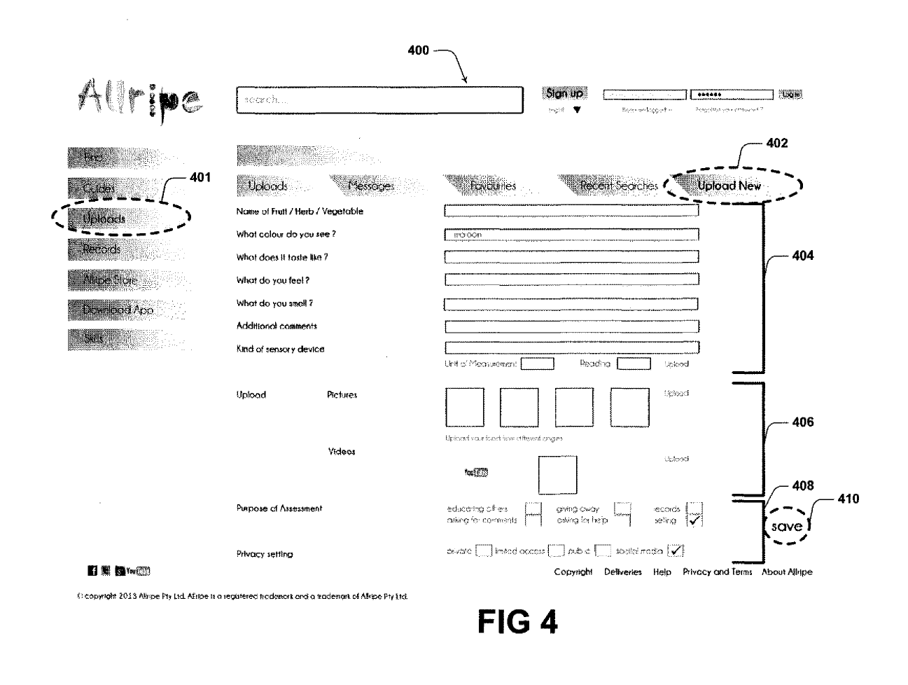

At step 304, a user logs into their established user account by entering the username and password set in step 302. Upon logging in, the user is presented with a number of selectable options, as illustrated for example in the webpage 400 shown in FIG. 4 for assessing a fruit or vegetable. In this exemplary embodiment, a number of vertically stacked selectable navigation tabs are included on the left-hand side. Amongst several other tabs is a tab labelled ‘Uploads’ 401. Selecting the ‘Uploads’ tab navigates the user to the page illustrated. At the top of the page a number of horizontally disposed tabs allow a user to navigate between sub-pages offering different options including viewing previous uploads (‘Uploads tab’), viewing and sending messages to other users (‘Messages’ tab), viewing and setting favourite settings for uploads (‘Favourites’ tab), viewing recent searches (‘Recent Searches’ tab) and uploading new food assessment data through the ‘Upload New’ tab 402. Selecting the ‘Upload New’ tab presents the user with the input fields displayed in FIG. 4.

Returning to FIG. 3, at step 306, a user then enters food assessment data by completing one or more corresponding input fields provided at interface 203. In the case of FIG. 4, a number of text fields 404, upload buttons 406 and check boxes 408 are provided for entering information. Text fields 404 allow a user to upload new food assessment data in the form of descriptive text. Upload buttons 406 allow a user to upload photos and videos relating to food items or the assessment of those food items. Check boxes 408 allow a user to add further information such as a purpose of the assessment and privacy settings.

Webpage 400 provides capability for a user to enter assessment information including the name of the fruit/herb/vegetable being assessed, the colour, taste, feel and smell of the food, as well as additional comments, device information (in the case a measuring device or instrument is used), photographs and videos. It will be appreciated that in other embodiments additional fields are provided to allow input of other food characteristics including size/volume, flavour, acidity, sugar content, fat content, ripeness, firmness, freshness and predicted longevity.

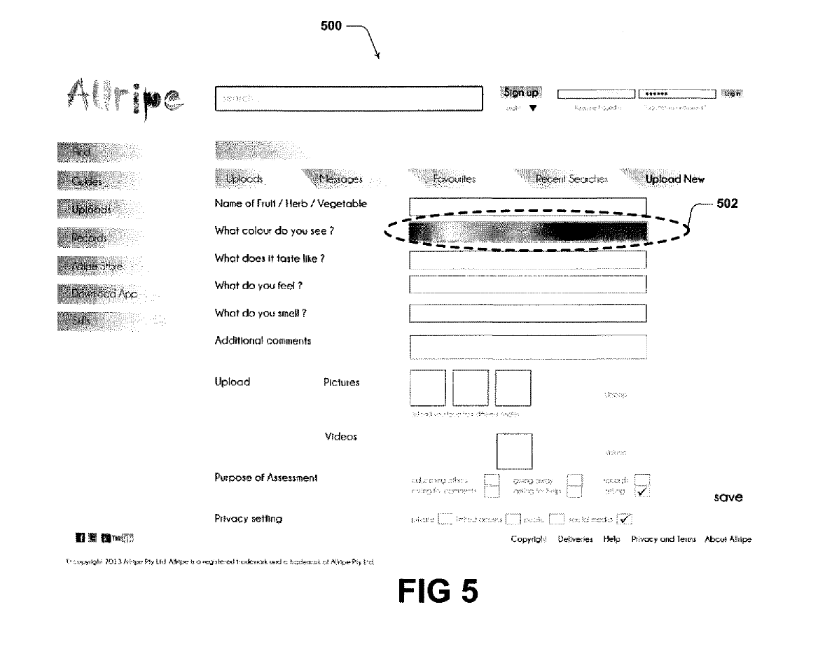

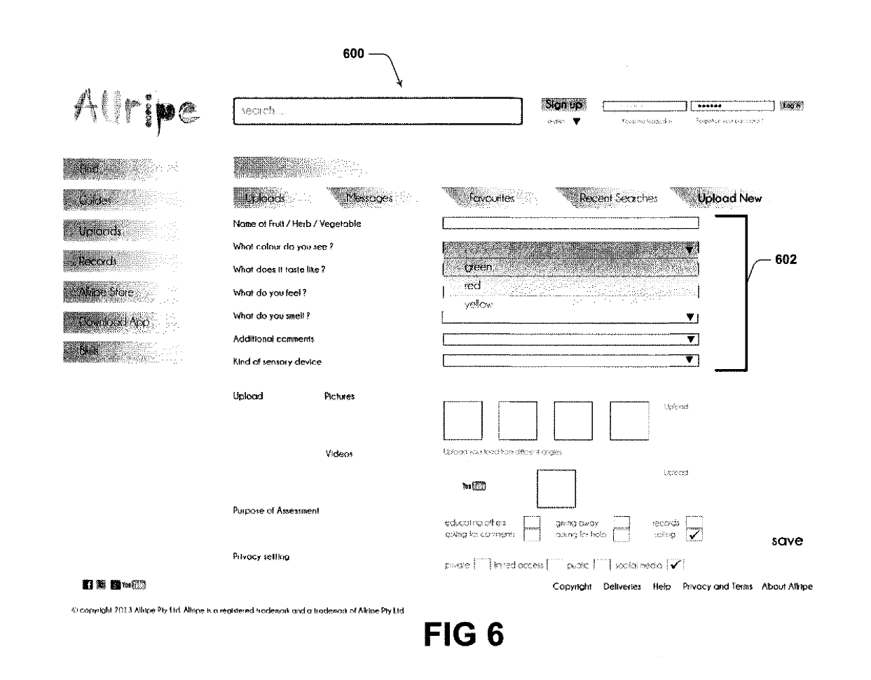

FIG. 4 illustrates input fields in the form of text fields, upload buttons and check boxes. It will be appreciated that other types of input fields are able to be utilised. Referring now to FIG. 5, there is illustrated a second embodiment webpage 500 including a colour input field 502 that comprises a colour bar. In this embodiment, the assessed colour of the fruit/herb/vegetable is chosen by a user selecting a position along the colour bar that corresponds closely to the colour of the fruit/herb/vegetable. FIG. 6 illustrates a third embodiment webpage 600 including drop-down menus 602 which are populated with preselected options.

Referring again to FIG. 3, finally, at step 308, the user submits the entered food assessment data. In the example of FIG. 4, the user selects the ‘save’ button 410 to upload the food assessment data to database 210. Each time a user submits the entered food assessment data, a sample record for the sample food item is created and stored in database 210. Similarly, a sample record for a sample food item is created upon receipt of quantitative data from an assessment device 214. In some embodiments, the data uploaded each time ‘save’ button 410 is selected is collectively stored as an individual assessment.

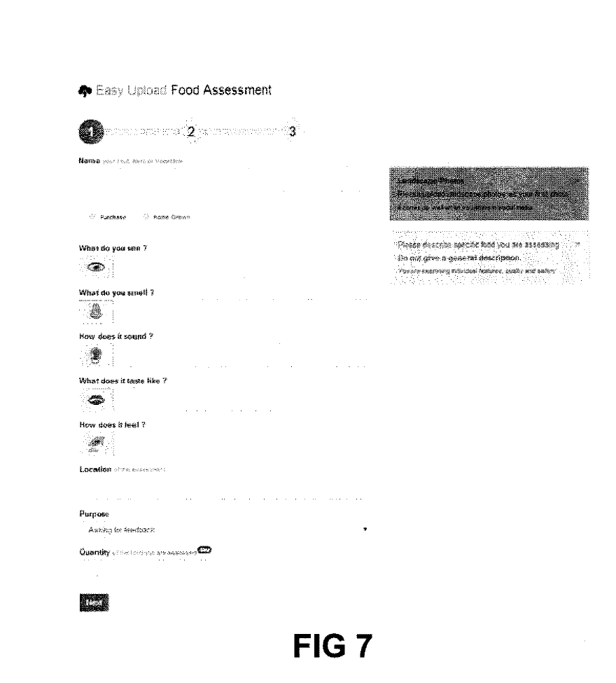

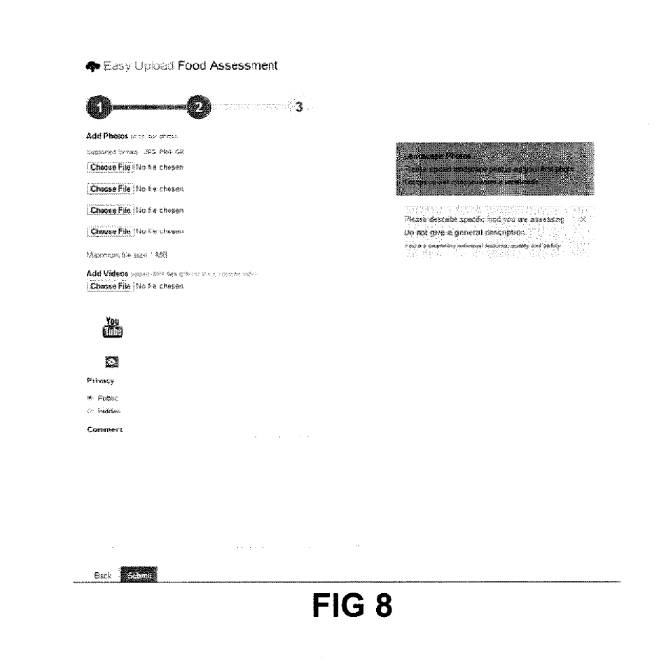

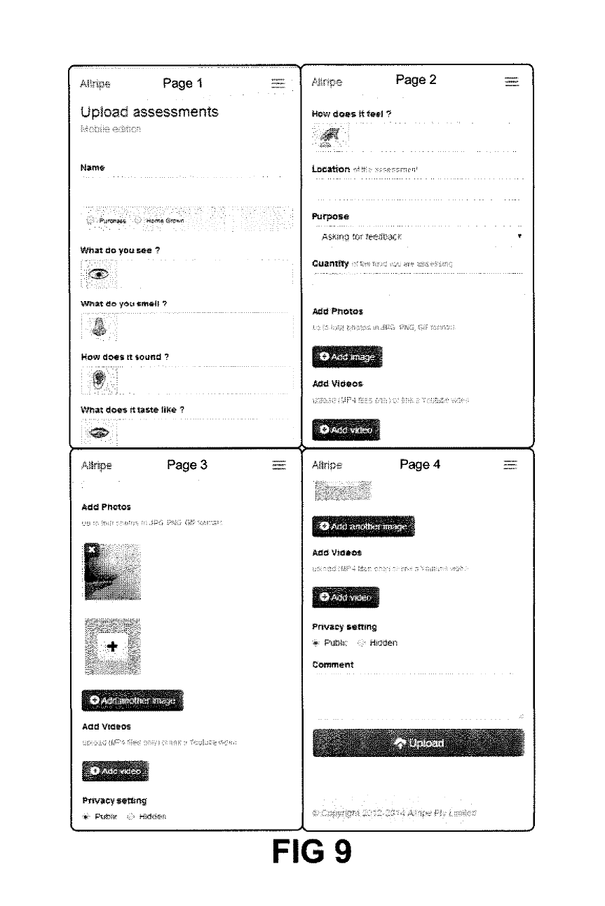

Further exemplary assessment data upload web pages are illustrated in FIGS. 7 and 8. The web pages illustrated in FIGS. 4 to 8 are adapted for a personal computer or laptop computer. Exemplary assessment data upload pages of a mobile ‘App’ for a mobile device (such as a Smartphone or tablet computer) are illustrated in FIG. 9.

The received input data is pre-processed as per step 104c in FIG. 1 and filtered for impurities or meaningless information. The processed input data is stored in database 210. The stored food assessment data defines a sample record for the particular food item or items. Predefined benchmarks based on previous food assessment data may also be stored in database 210.

### Analysis of Food Assessment Data

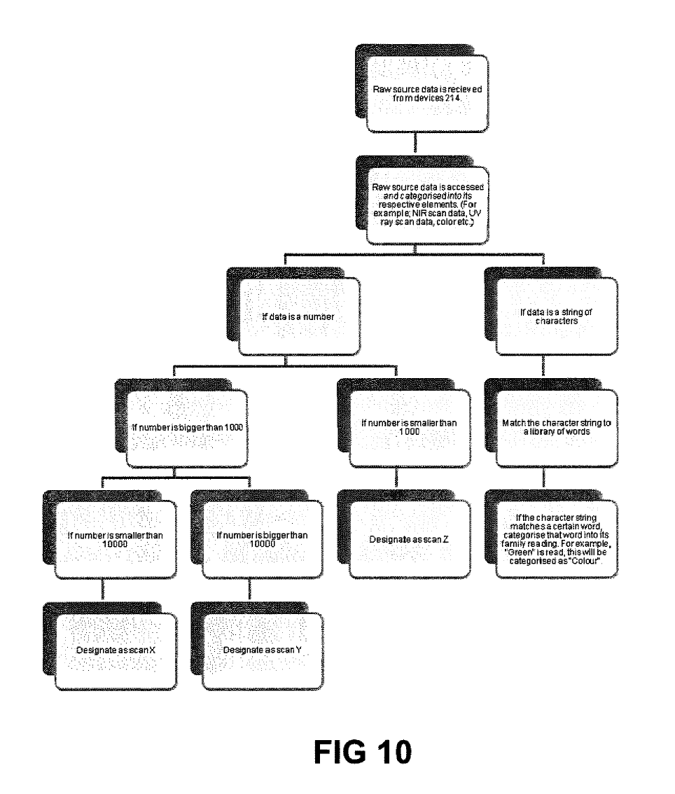

Following storage in database 210, the uploaded food assessment data is processed by processor 205. Processor 205 is configured to implement a food assessment model that is selectively responsive to the sample record and the other records to provide an indication of one or more characteristics of the sample food item. In the data processing step, the data are associated with specific attributes relating to the source of the data, type of data, input method and relevant food item or items being assessed. Upon receipt of input data collected in the field by devices 215, processor 205 executes one or more sorting algorithms which determines the data type and associates the data with appropriate attributes. An example sorting algorithm is illustrated schematically in FIG. 10.

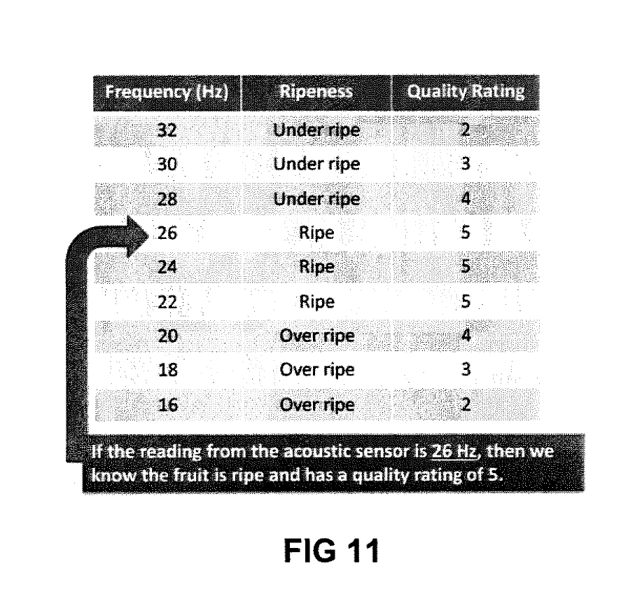

Database 210 stores look-up tables relating input data of various sensor instruments to corresponding food assessment data values. Processor 205 is configured to, upon input of raw sensor data, identify the input data type and automatically access the appropriate look-up table in database 210 to convert the raw sensor data (e.g. frequency, voltage or wavelength data) to assessment data suitable for model calculations. In this manner, input data of different forms are able to be input and converted to a common data form that is simpler for a user to understand and is easily applied directly in the model calculations. Referring to FIG. 11, there is illustrated an example look-up table utilised by processor 205 for relating input frequency data to ripeness and quality rating.

If the input sensor data is of a type that is not recognised by processor 205, interface 203 prompts a user to input an attribute for that data, associate the data with a particular look-up table or add a new look-up table or conversion to database 210.

The table below lists exemplary types and sources of input data and the various attributes with which the data are associated upon upload. Example analytic tools that are applied to the data and the associated useful output that the data is able to produce are also shown.

| Data Source | Data Type | Input Method | Analytic Tools | Outputs |
|---|---|---|---|---|
| Multiples | Readings | Website entry | Databases | Display of |
| sensors | Pictures | App entry | Look Up | food |
| Photographic | Video footage | Wi-Fi connection | Tables | information |
| camera | Frequencies | User entered | Signal | Plots & maps |
| Video camera | Electronic | information | conversion | Data |
| Photodiodes | signals | Device/instrument | Signal | visualisation |
| Ultraviolet (UV) | Digital signals | uploads | filtering | Website |
| sensors | Numbers | Remote entry | Statistical | display |
| Visible | Code | Bluetooth | analysis | Application |
| spectrum | Abbreviations | connection | Predictive | display |
| sensors | Various units | Image uploads | modelling | Read-out |
| Near Infrared | of measure | Frequency | Composite | Phone |
| sensors | Text | uploads | modelling | message, |
| Mid Infrared | descriptions | Other signal | Pathogenic | such as SMS |
| sensors | Sense-data | uploads | finding | Email |
| Far infrared | Sense- | File upload | Pathogenic | message |
| sensors | impressions | Image input | identification | Print out |
| Piezoelectric | Template | Coding | Comparisons | Digital file |
| detectors | uploads | Colour palette | Analysis | Various |
| Quartz Crystal | Sensor | Drop-down | Equations | document |
| Microbalance | readings | menus | Visualisations | formats |
| sensors | | Tabs, Text fields | Mapping | Downloads |
| Magnetoelastic | | Text fields | Data values | Text, |
| sensors | | Tick boxes | Frequency | numeric, |
| Lab-On-Chip | | | conversions | visual & |
| sensors, | | | Algorithms | other |
| Biochemical | | | | sensory |
| Phages & | | | | outputs |
| chemical layers | | | | Audio & |
| Penetrometer, | | | | podcast |
| refractometer, | | | | Icon displays |
| E-Noses, Lab | | | | Table & tab |
| equipment & | | | | displays |
| other | | | | Automatic |
| sensors/detectors | | | | population of |
| | | | | fields |
| | | | | Presentation, |
| | | | | Meaning |

In addition to the attributes listed above, a wide variety of other attributes are able to be associated with the uploaded assessment data including, the relevant type or types of food assessed, the location of the food or food assessment (country, state, region etc.), date, time, climate (rainfall, temperature etc.), season, the grower/farmer/manufacturer of the food, the user who contributed the data and the purpose of the data (e.g. safety, quality informative).

In one embodiment, the process of inputting and processing the food data is performed in five main stages performed by processor 205, as shown in FIG. 9. At stage 1, the user provides input food assessment data. Upon the input, at a second stage 2A, processor 205 extracts the name of the food (fruits/herbs/vegetables) from the input data. Based on the food name, processor 205 searches benchmarks which are stored in database 210 to acquire information about the food. At a stage 2B, the information returned contains the different possible stages of the food (e.g. Mango, unripe) and the corresponding ratings of the different outcome parameters. All information regarding the different benchmark stages of the same food is stored in the database for later use.

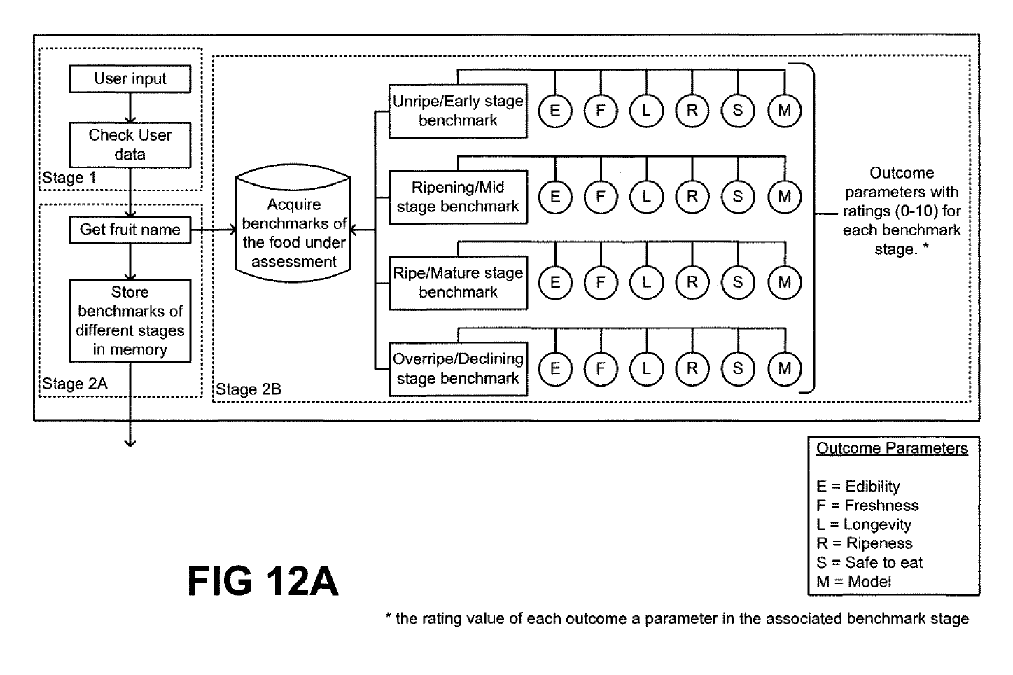

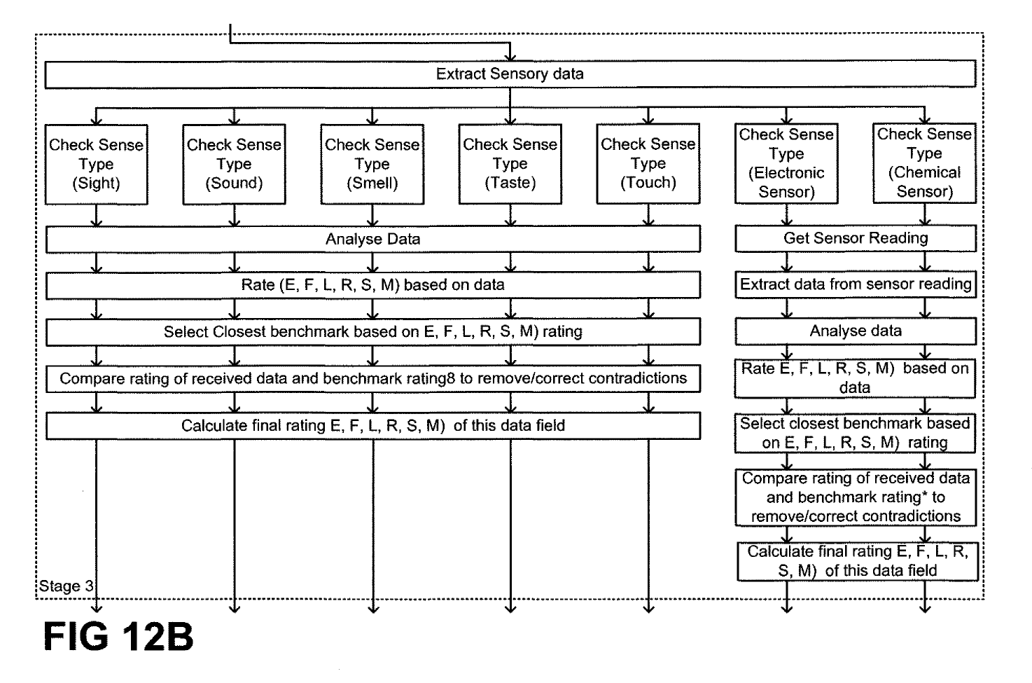

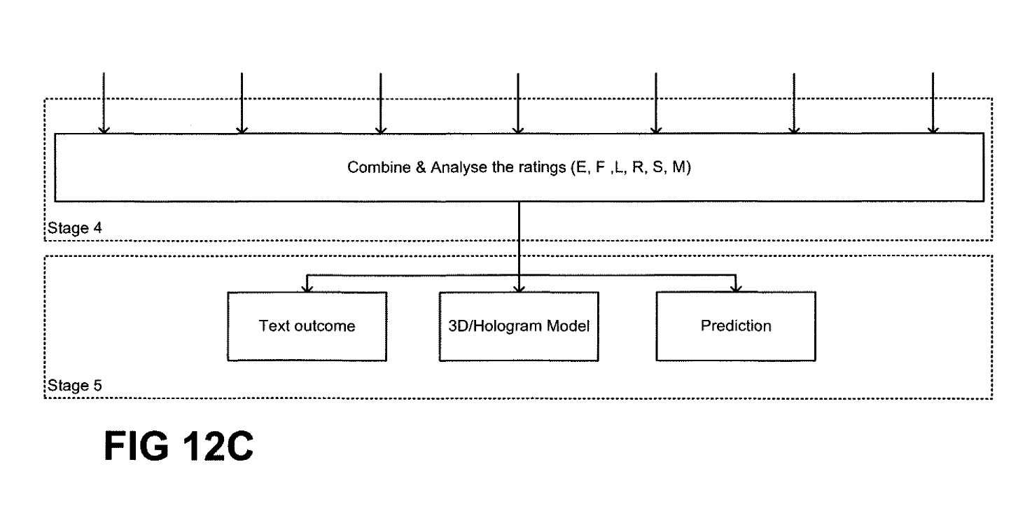

Next, at a third stage, processor 205 extracts the sensory data received from the user and uses this as an input for a six-step composite analysis of the data. In the first step, processor 205 checks the type of sense which can be a textual data entered by a user or could be data from an electronic or chemical device. The data received from a text field entered by the user are treated differently from device data. In case of textual data, processor 205 takes all the different sense data and analyses them separately, as per FIG. 12B.

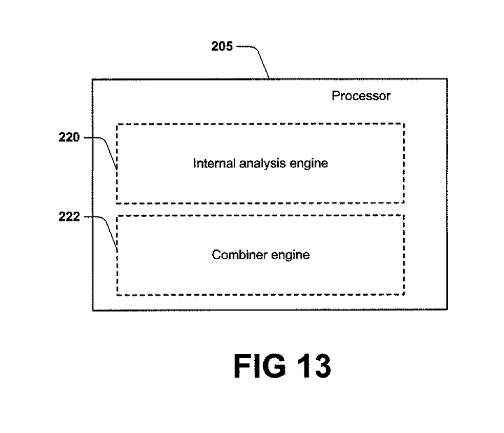

Referring to FIG. 13, processor 205 includes functionality to perform as an internal analysis engine 220 for extracting device data (electronic/chemical) from the sensor reading and performing data analysis. After analysing the data, processor 205 calculates the different values for each of the outcome parameters for the food under assessment. Based on the rating, engine 220 chooses the closest benchmark which was previously stored in database 210.

In the next step of stage 3, the values of the outcome parameters for the food under assessment and the outcome parameter values of the corresponding selected benchmark are compared. Any contradictory data is removed or corrected depending on the quality of the data. After the correction step, another set of the outcome parameters (E, F, L, R, S, and M) is calculated.

At stage 4, the outcome parameters are passed to processor 205, which is now configured to function as a combiner engine 222, as shown in FIG. 13. The combiner engine 222 receives all the different outcome parameters calculated for each sense or sensor. Combiner engine 222 takes all the input ratings from different sensors and sense data entered by the user and calculates the expected values of each rating variable. By way of example, to calculate an edibility rating (E), combiner engine 222 takes the value of E from each sense data or sensor data and calculates the expected value. For example, with 5 sense data and 2 sensors the expected mean may be calculated by the following equation.

Expected value=Xₛₛ₁* 1/7+Xₛₛ₂* 1/7+Xₛₛ₃* 1/7+Xₛₛ₄* 1/7+Xₛₛ₅* 1/7+Xₛᵣ₁* 1/7+Xₛᵣ₂* 1/7

In the above equation different sense data are represented by ‘ss’ and sensor data is represented by ‘sr’. Since there are 7 points of input in the present embodiment, each input is equally weighted by a factor of 1/7 to calculate the expected value.

Based on the expected values of outcome parameters, processor 205 generates a composite output food assessment model including four main outputs:

Text outcome

Abstract model

Multi-sensory model

Prediction

The model may take the form of a hologram or a 3D model such as a 3D image or representation of the food item. The prediction provided may include a predicted product keep date, predicted ripeness, predicted wholesale price or a predicted retail price.

Also considered is the geographic location, season, climate along with the expected rating values while generating the text outcomes. This is because; some of the text data (e.g. prediction of the product keep date or ripeness longevity) depends on the actual climate and season where the food is currently located.

The text outcome contains the freshness, safety, ripeness and edibility status of the food which can be printed as a result format on the screen. The outcome parameters can be used along with the variable time to generate predictions regarding the food. The model variable in the outcome rating is used to create a 3D/Hologram model.

### Model Generation

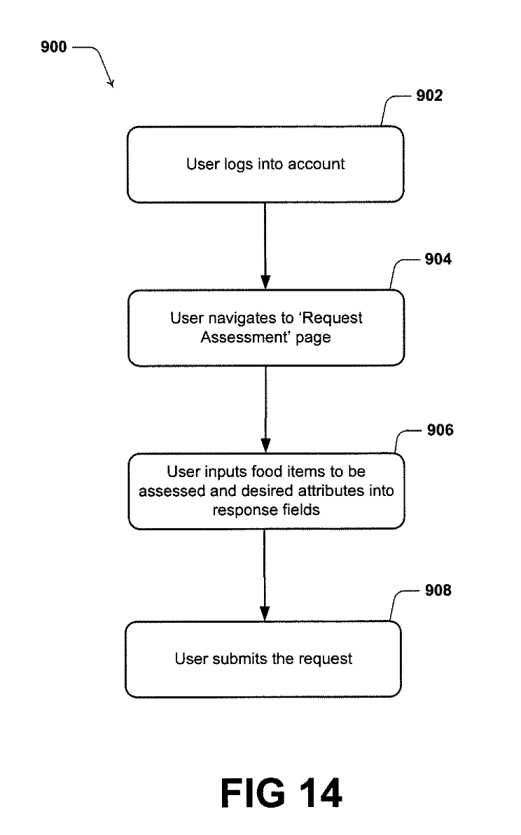

A user wishing to seek an assessment of a particular food item follows the steps of method 900 set out in FIG. 14. At step 902, the user logs into their user account on server 202. At step 904, the user navigates to a ‘Request Assessment’ webpage through interface 203. In embodiments utilising a mobile device App, at step 904, the user navigates to a ‘Request Assessment’ screen by appropriate manipulation of the device inputs. At step 906, the user inputs request criteria including one or more food items for assessment and attributes relevant to the desired assessment into response fields provided on the webpage. The attributes entered by the user act as filter parameters for identifying the relevant food assessment data stored in database 210. By way of example, a user request may include prompts and responses such as <‘food type’=green apples>, <‘location’=New South Wales, Australia>, <‘season’=summer>.

Once the user has completed all desired fields, at step 908 the user selects a ‘submit’ button in interface 203 to submit the request to server 202.

In response to the submitted user request, processor 205 performs the steps illustrated in FIGS. 12A to 12C. This includes, identifying the subset of food assessment data or sample record stored in database 210 which satisfy all the request criteria. Processor 205 then combines the subset of data and applies various weightings to the data based on the attributes to produce an assessment model of the food item. The food assessment data is compared with the previous assessment data and data relating to similar items to assess the quality and safety of the food items being assessed.

The composite model can be presented in different formats depending on the variables used and the type of food under assessment. Three exemplary types of composite models, namely the composite line model, composite pie model and composite map model.

### Pie Model

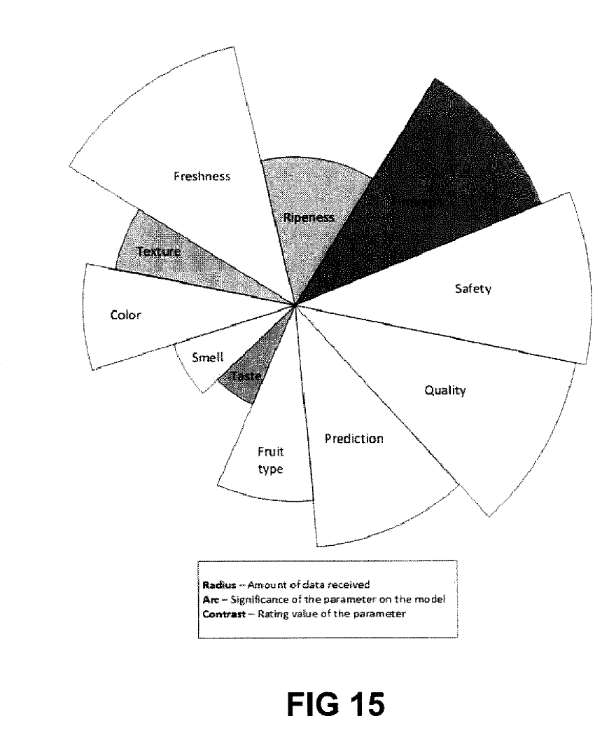

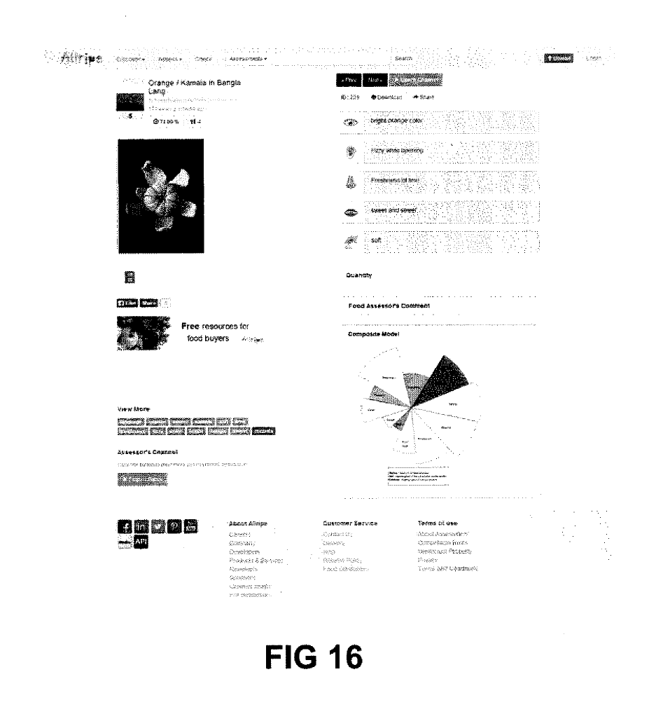

A composite pie assessment model is illustrated in FIG. 15. A corresponding online assessment report including the pie model is illustrated in FIG. 16.

The composite pie model incorporates a pie chart to represent the food under assessment. Each of the slices are different variables chosen from both outcome parameters (e.g. Freshness) and input variables entered by users (e.g. Smell). The size of each slice depends on the value received on that variable. The radius of each slice changes according to the amount of data received in those particular variables, whereas the arc angle depends on the significance of the variable on the model. The greyscale contrast of each slice is determined by the actual value of the variable.

### Map Model

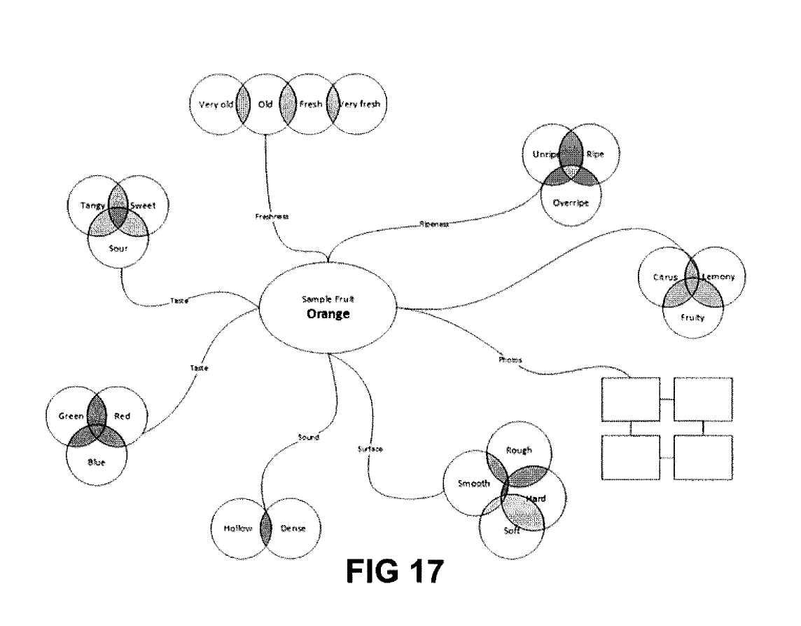

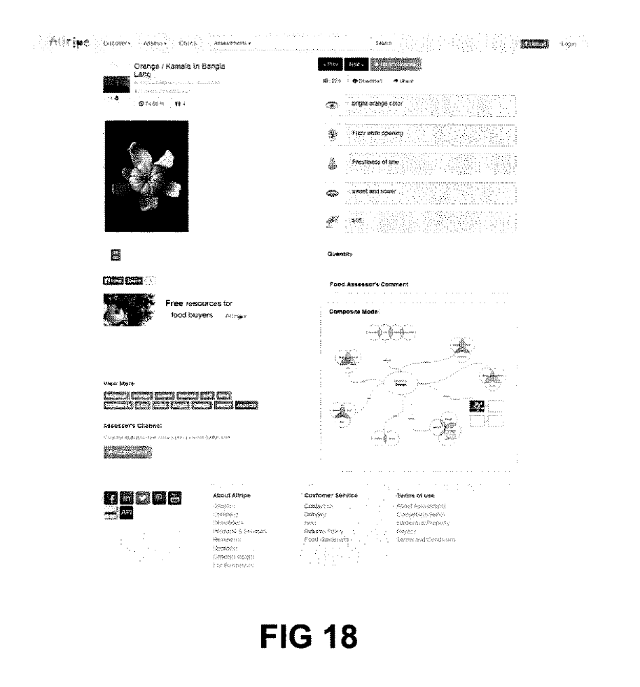

The composite assessment model can also be shown as a map, as illustrated in FIG. 17. An exemplary online report including a map assessment model is illustrated in FIG. 18. In the map model, the central region indicates the food under assessment. Each of the branches which extend from the central region illustrates a variable and an end of the branch intersects with the corresponding condition or rating from a group of ratings. In the case of some variables such as colour, the common portions of the different intersections determine the condition of the food under assessment.

### Line Model

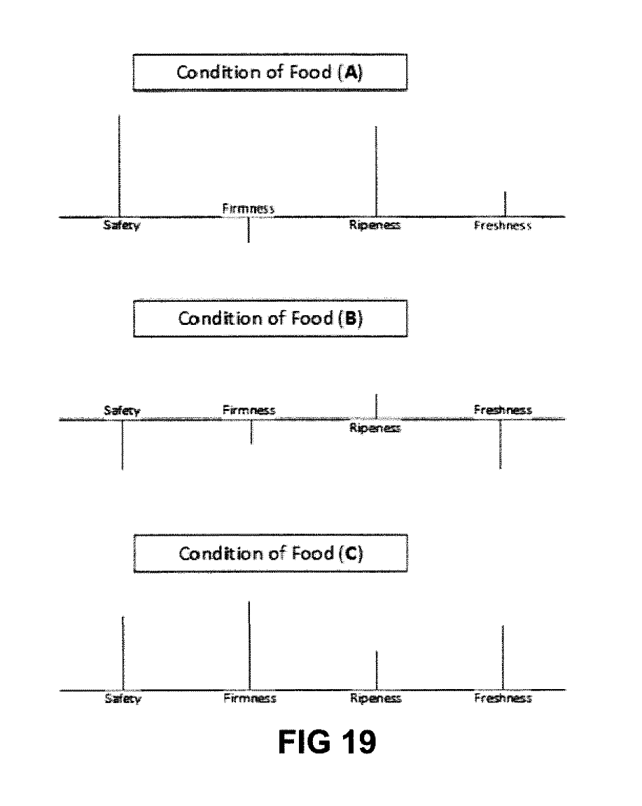

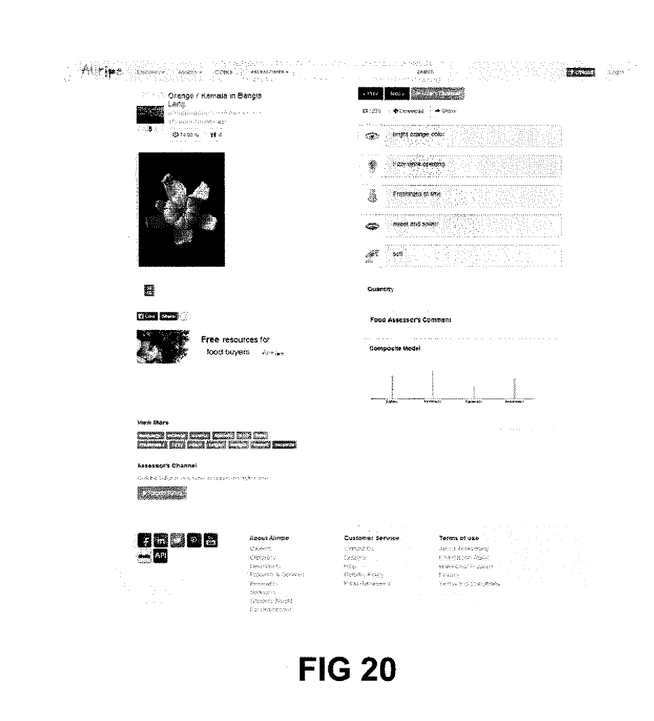

Composite assessment model of the outcome parameters can be presented using a line diagram, as illustrated in FIG. 19. An exemplary online assessment report incorporating a line model is illustrated in FIG. 20.

In the line model, each line represents a variable. The illustrated variables under consideration are safety, freshness, firmness and ripeness of the food under assessment. The length of the line along the vertical y-axis changes according to the value of the parameters, which can be positive, negative or even zero.

In some embodiments, the assessment model includes both a static version and a dynamic version of the food item being assessed. The static version represents an assessment of the food item at the time the request was made. The dynamic version provides an up-to-date assessment of the food that is updated regularly at certain times to include new data (both user-uploaded and instrument-based recordings) that is added to database 210.

In producing the assessment model, processor 205 performs various algorithms and solves various equations to enable the food assessment data to be transformed into, amongst other forms, probable growth paths of the food item (in the case of growable foods). The algorithms use features to predict an overall model and possible ways the food item can be further developed, grown or manufactured from a specific point.

The algorithms used by processor 205 include emergent algorithms, performing numerical solution of differential-algebraic equations or differential equations, least squares regression analysis, multiple regression analysis and solving multidimensional arrays (structure arrays, cell arrays, numeric arrays and/or character arrays).

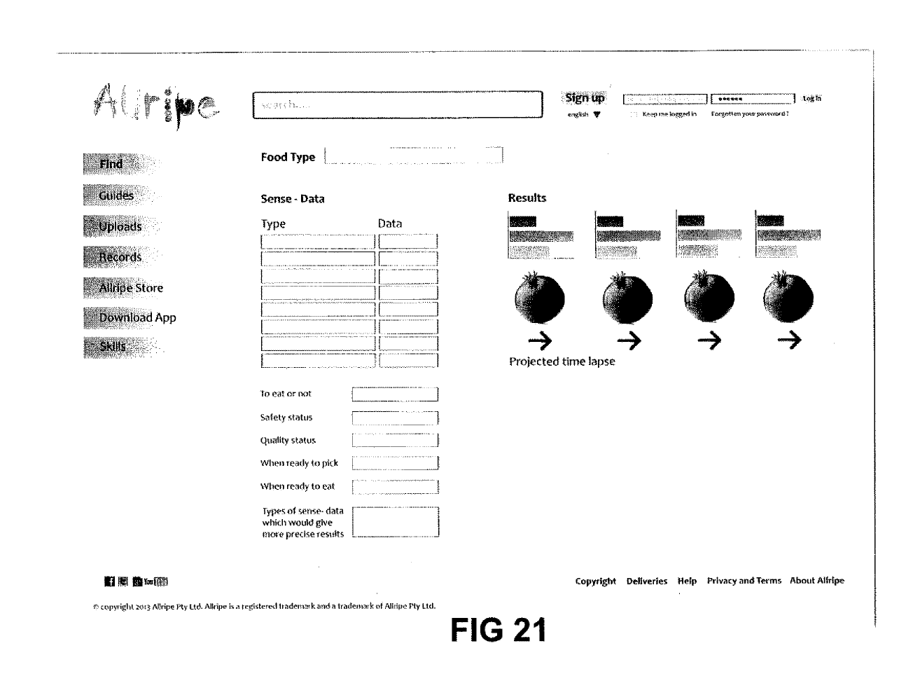

The presentation of data in the assessment model includes mathematical representation (for example, percentages, scores and ratings), time-sequence (for example, data plots and data graphs) or graphical representation (for example, texture mapping, wireframe models, display of food colour, shape and surface, solid modelling, time coded photographs and videos). An exemplary webpage illustrating time-lapse results data is shown in FIG. 21. The right side of FIG. 21 illustrates four panels of exemplary data relating to a food item taken at different time intervals.

The assessment model is also capable of producing a computer simulation of the physical food item, defined by its characteristics which are acquired by both human senses and/or assessment devices 215.

Sense data & analysed data serve as input signals for the static & dynamic simulations' models. Using sense data in a mathematical model to represent the features, characteristics & quality of food. Emergent structure of organic or biological form derived from different & varying combinations of sense-data.

The generated assessment model may include, amongst other information, estimations of the ripeness, longevity, nutritional content, quality, safety, advice and comparisons in quality and safety with similar food items. The information included in the model is displayed to the user in the form of graphs, text, pictures and videos. The model also includes supplementary information such as relevant professional reviews of the food items, photos, food diagrams and videos (e.g. recorded interviews of professionals about the particular food item or related food items). In some embodiments, the model includes a suggested selling price for the food item or locations to purchase the food items.

In some embodiments, the assessment model also includes one or more ratings of the food item such as a quality rating, safety rating or health benefit rating of the food item or items assessed. In one embodiment, the ratings are a numerical rating out of 100.

In another embodiment, the ratings are text such as ‘good’, ‘average’ and ‘bad’. The ratings are calculated based on a number of factors including the amount of positive user feedback, comparisons of recent food assessment data with earlier assessment data and comparisons of measured food properties (such as acidity levels) with known benchmark properties.

In one embodiment, the assessment model and/or ratings include an associated reliability estimation indicating the reliability of the assessment or rating. A model or rating of foods that is derived from less food assessment data will have a lower reliability than a model or rating of foods derived using greater food assessment data. In one embodiment, the user account information is also considered in calculating the reliability wherein the past experience of a user and the user's status (professional or non-professional) are factors, as well as the number of different users that have contributed to the assessment data for a food. By way of example, an assessment model or rating derived from food assessment data contributed by a large number of different users will have a higher reliability than an assessment model or rating derived from food assessment data contributed by a small number of different users. Similarly, an assessment model or rating derived from food assessment data contributed by professional users will have a higher reliability than an assessment model or rating derived from food assessment data contributed by non-professional users.

Once an assessment model is generated, a user is able to produce an assessment report summarising certain information presented in the model. Interface 203 includes prompts to allow a user to select data from a model to include in an assessment report and submit a request to have the report generated. In one embodiment, assessment reports are generated as a PDF file and emailed to an email address associated with the user's account. In another embodiment, assessment reports are generated as HTML-based files that can be implemented into a webpage or shared on a social media network such as Facebook. In another embodiment, the assessment model is displayed in a virtual reality context. In a further embodiment, the food composite model is displayed on a display such as a television or screen of a refrigerator.

In one embodiment, processor 205 generates an inline frame in association with an assessment report. The inline frame includes an inline frame tag (<iframe> tag) that a user can access to embed the generated assessment report within a website. In this embodiment, the assessment report includes text and/or other media such as images and embedded video content.

Parties interested in the assessment report are able to embed the report in their website through payment of a fee or under a license agreement. Interested parties include supermarkets, retailers, wholesalers, food distributors, micro-food producers, food gardeners, online marketplaces, food re-sellers, home delivered grocery suppliers, farmers and manufacturers of food items being assessed. In other embodiments, interested parties are able to embed the generated assessment report into a website free of charge and license-free. In some embodiments, the generated assessment report includes one or more advertisements. In one embodiment, the advertisements are relevant to the food item being assessed in the assessment report.

In addition to producing assessment models and assessment reports, interface 203 also allows a user to search for stored assessment information on food items of interest. This is possible by inputting the sample food item as a virtual food item with virtual characteristics. Referring again to FIGS. 4 to 6, a search field is provided at the top of the webpages. Assessment information includes stored sample records of various food items including individual food assessments or reviews, assessment models and previously generated assessment reports. A user is able to enter virtual characteristics as search terms and issue a search for assessment information on relevant food items. Processor 205 compares the entered search terms with the attributes of data stored in database 210 and returns assessment information having attributes relevant to the specified search terms. By way of example, a user may enter search terms <safety, red meat, New South Wales, assessment reports> to return a list of assessment reports that relate to the safety of red meat in New South Wales, Australia. The data returned from the search is able to be exported to other parties and data servers either as a free service or for a fee.

It will be appreciated that in various embodiments, the invention is able to be implemented on different computer platforms. In some embodiments, interface 203 is accessed by client terminals 204 in the form of Smartphones such as the Apple iPhone or Google Android based phones. In other embodiments client terminals 204 include personal computers and interface 203 is accessed through a web browser. In each embodiment, client terminal processors 211 render the data input and output through interface 203 in a format that is compatible with the relevant computer platform in use. For example, in the case where a user accesses interface 203 through an Apple iPhone, interface 203 is rendered with a screen size compatible with the iPhone and having input prompts suitable for touch screen user inputs.

### Improved Use of a Computer

In the present invention, sorting algorithms and automatic detection of data formats are utilised by processor 205. Further, upon detection of the data format, automatic conversion of data to a common format is performed by processor 205. These technical processing steps allow a number of individual and distinct food assessments, in the form of food assessment data received from a variety of different sources (both electronic sensors and human observations), to be combined and a composite assessment model to be produced for a particular one or more sample food items. Generation of an actual assessment model again involves the technical steps of sourcing, combining and weighting the various food assessment data, stored in database 210, based on input filter parameters entered by a user through interface 203.

Food assessment reports are generated by processor 205 and are associated with inline frame tags for embedding the reports in websites of interested parties.

### CONCLUSIONS AND INTERPRETATION

It will be appreciated that the disclosure above provides various significant computer implemented systems and methods for analysing food assessment data. The assessment is derived from the modelling of physical entities sourced from a combination of various and differing sense (human inputs) & sensor data (instrument and device inputs). Embodiments of the present invention provide a substantial and credible use in enabling people to source food assessment data from a wider range of data sources than has been possible in the past. The invention enables interested parties to use food assessment analytics more easily and receive more meaningful information and recommendations on a specific food's quality and safety. Embodiments of the present invention enable an assessment based on a combination of artificial sensors as well as human senses to produce a more detailed and more holistic model of a food than is currently available.

From the generated assessment model, useful quality and safety information can be extracted and meaningful recommendations can be provided concerning the food under investigation so that a user can make better decisions on food concerning its safety and quality. The data, analyses and recommendations are presented to users so they can understand the food's condition across multiple sensory fronts.

The present invention is able to be used by a much broader range of participants in the food supply chain than has traditionally been possible. Through use of interface 203, food assessment is accessible to more food producers, food supply chain intermediaries, retailers of food, eateries & consumers than at present. These parties are able to assess their food more quickly, easily, accurately and affordably.

The inventors envisage that the present invention will be applicable to other fields of assessment other than food, including by way of example, wood, steel and other construction materials, wine, soils, air, water, plants, drinks, medicines and fertilisers.

Unless specifically stated otherwise, as apparent from the following discussions, it is appreciated that throughout the specification discussions utilizing terms such as “processing,” “computing,” “calculating,” “determining”, analysing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer” or a “computing machine” or a “computing platform” may include one or more processors.

The methodologies described herein are, in one embodiment, performable by one or more processors that accept computer-readable (also called machine-readable) code containing a set of instructions that when executed by one or more of the processors carry out at least one of the methods described herein. Any processor capable of executing a set of instructions (sequential or otherwise) that specify actions to be taken are included. Thus, one example is a typical processing system that includes one or more processors. Each processor may include one or more of a CPU, a graphics processing unit, and a programmable DSP unit. The processing system further may include a memory subsystem including main RAM and/or a static RAM, and/or ROM. A bus subsystem may be included for communicating between the components. The processing system further may be a distributed processing system with processors coupled by a network. If the processing system requires a display, such a display may be included, e.g., a liquid crystal display (LCD) or a cathode ray tube (CRT) display. If manual data entry is required, the processing system also includes an input device such as one or more of an alphanumeric input unit such as a keyboard, a pointing control device such as a mouse, and so forth. The term memory unit as used herein, if clear from the context and unless explicitly stated otherwise, also encompasses a storage system such as a disk drive unit. The processing system in some configurations may include a sound output device, and a network interface device. The memory subsystem thus includes a computer-readable carrier medium that carries computer-readable code (e.g., software) including a set of instructions to cause performing, when executed by one or more processors, one of more of the methods described herein. Note that when the method includes several elements, e.g., several steps, no ordering of such elements is implied, unless specifically stated. The software may reside in the hard disk, or may also reside, completely or at least partially, within the RAM and/or within the processor during execution thereof by the computer system. Thus, the memory and the processor also constitute computer-readable carrier medium carrying computer-readable code.

Furthermore, a computer-readable carrier medium may form, or be included in a computer program product.

In alternative embodiments, the one or more processors operate as a standalone device or may be connected, e.g., networked to other processor(s), in a networked deployment, the one or more processors may operate in the capacity of a server or a user machine in server-user network environment, or as a peer machine in a peer-to-peer or distributed network environment. The one or more processors may form a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine.

Note that while diagrams only show a single processor and a single memory that carries the computer-readable code, those in the art will understand that many of the components described above are included, but not explicitly shown or described in order not to obscure the inventive aspect. For example, while only a single machine is illustrated, the term “machine” shall also be taken to include any collection of machines that individually or jointly execute a set (or multiple sets) of instructions to perform any one or more of the methodologies discussed herein.

Thus, one embodiment of each of the methods described herein is in the form of a computer-readable carrier medium carrying a set of instructions, e.g., a computer program that is for execution on one or more processors, e.g., one or more processors that are part of web server arrangement. Thus, as will be appreciated by those skilled in the art, embodiments of the present invention may be embodied as a method, an apparatus such as a special purpose apparatus, an apparatus such as a data processing system, or a computer-readable carrier medium, e.g., a computer program product. The computer-readable carrier medium carries computer readable code including a set of instructions that when executed on one or more processors cause the processor or processors to implement a method. Accordingly, aspects of the present invention may take the form of a method, an entirely hardware embodiment, an entirely software embodiment or an embodiment combining software and hardware aspects. Furthermore, the present invention may take the form of carrier medium (e.g., a computer program product on a computer-readable storage medium) carrying computer-readable program code embodied in the medium.

The software may further be transmitted or received over a network via a network interface device. While the carrier medium is shown in an exemplary embodiment to be a single medium, the term “carrier medium” should be taken to include a single medium or multiple media (e.g., a centralized or distributed database, and/or associated caches and servers) that store the one or more sets of instructions. The term “carrier medium” shall also be taken to include any medium that is capable of storing, encoding or carrying a set of instructions for execution by one or more of the processors and that cause the one or more processors to perform any one or more of the methodologies of the present invention. A carrier medium may take many forms, including but not limited to, non-volatile media, volatile media, and transmission media. Non-volatile media includes, for example, optical, magnetic disks, and magneto-optical disks. Volatile media includes dynamic memory, such as main memory. Transmission media includes coaxial cables, copper wire and fibre optics, including the wires that comprise a bus subsystem. Transmission media also may also take the form of acoustic or light waves, such as those generated during radio wave and infrared data communications. For example, the term “carrier medium” shall accordingly be taken to included, but not be limited to, solid-state memories, a computer product embodied in optical and magnetic media; a medium bearing a propagated signal detectable by at least one processor of one or more processors and representing a set of instructions that, when executed, implement a method; and a transmission medium in a network bearing a propagated signal detectable by at least one processor of the one or more processors and representing the set of instructions.

It will be understood that the steps of methods discussed are performed in one embodiment by an appropriate processor (or processors) of a processing (i.e., computer) system executing instructions (computer-readable code) stored in storage. It will also be understood that the invention is not limited to any particular implementation or programming technique and that the invention may be implemented using any appropriate techniques for implementing the functionality described herein. The invention is not limited to any particular programming language or operating system.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

What is claimed is:

1. A computer implemented method to process food assessment data, the method including:
a) providing an interface to enable input of food assessment data;
b) maintaining a database to store the assessment data;
c) configuring a processor to analyse at least a subset of the assessment data based on one or more of the type and source of the assessment data to produce a food assessment model indicating the safety and/or quality of a selected one or more food items.

2. A computer implemented method according to claim 1 wherein the processor is also configured to identify the type of input food assessment data and, in response, convert the data to a second data format that is independent of the data type.

3. A computer implemented method according to claim 1 wherein the processor is also configured to identify the source of input food assessment data and, in response, convert the data to a second data format that is independent of the data source.

4. A computer implemented method according to claim 1 wherein the processor is configured to assign attributes to the input food assessment data based on a type and a source of the data.

5. A computer implemented method according to claim 4 wherein the processor separately analyses the assessment data based on the type and/or the source of the data.

6. A computer implemented method according to claim 5 wherein the type of the data includes human sense data and instrument sensor data.

7. A computer implemented method according to claim 5 wherein the source of the data includes human input or electronic sensor input.

8. A computer implemented method according to claim 7 including the step of:
c) i) calculating one or more food assessment parameters including the Edibility(E), Freshness(F), Longevity(L), Ripeness(R) or Safeness to eat(S) of the food item.

9. A computer implemented method according to claim 8 including the step of:
c) ii) comparing the one or more food assessment parameters with a corresponding benchmark for that particular source or type.

10. A computer implemented method according to claim 9 including the step of:
c) iii) combining the comparisons of the one or more food assessment parameters for each data source and type.

11. A computer implemented method according to claim 4 wherein the processor applies a numerical weighting to the food assessment data based on the data attributes.

12. A computer implemented method according to claim 1 wherein the food assessment model includes one or more ratings indicative of the relative quality or safety of the one or more food items.

13. A computer implemented method according to claim 1 wherein the input food assessment data includes information indicative of one or more of a food's visual appearance, smell, size/volume, taste, flavour, colour, ripeness and firmness.

14. A computer implemented method according to claim 5 wherein the input data type includes text input, photographs, video, sensor data and spectrometer data.

15. A computer implemented method according to claim 1 wherein the food assessment model includes an assessment of whether a food is safe to eat, an assessment of the freshness of a food, or an assessment of the longevity of a food.

16. A computer implemented method according to claim 1 wherein the food assessment model includes an assessment report adapted to be shared through one or more websites.

17. A computer system configured to perform a method according to claim 1.

18. A non-transitive carrier medium carrying computer executable code that, when executed on a processor, causes the processor to perform a method according to claim 1.

19. A computer implemented method to provide food assessment data, the method including:
a) providing an interface to enable one or more users to input user assessments of one or more food items;
b) providing a communications module to receive device assessment data from measurement devices;
c) maintaining a database to store the user assessments and device assessment data;
d) upon a request by a user, configuring a processor to produce an assessment model of a preselected food item, the model being based on a combination of the user assessments and device assessment data for the preselected food item.

20. A system to provide an indication of one or more characteristics of a sample food item, the system including:
an interface to enable the creation of a sample record for the sample food item, the record including either or both of quantitative or qualitative data for the sample food item;
a database to store the sample record and other records for respective other sample food items; and
a processor to implement a food assessment model that is selectively responsive to the sample record and the other records to provide an indication of one or more characteristics of the sample food item.
